(19) United States
(12) Patent Application Publication
(10) Pub. No.: US 2004/0033578 A1
(43) Pub. Date: Feb. 19, 2004

(54) Cloning of a member of the serine-threonine-kinase family

(75) Inventors: Klaus Strebhardt, Frankfurt (DE); Helga Rubsamen-Waigmann, Bad Soden (DE); Uwe Holtrich, Kelkheim (DE)

Correspondence Address: KURT BRISCOE, NORRIS, MCLAUGHLIN & MARCUS, P.A., 220 EAST 42ND STREET, 30TH FLOOR, NEW YORK, NY, 10017, US

(73) Assignee: Klaus Strebhardt

(21) Appl. No.: 10/406,901

(22) Filed: Apr. 3, 2003

Related U.S. Application Data
(62) Division of application No. 09/634,443, filed on Aug. 8, 2000.
Application No. 09/634,443 is a division of application No. 09/198,122, filed on Nov. 23, 1998, now Pat. No. 6,180,380.
Application No. 09/198,122 is a division of application No. 08/601,014, filed on Feb. 23, 1996, now abandoned.
Application No. 08/601,014 is a 371 of international application No. PCT/EP94/02863, filed on Aug. 20, 1994.

(30) Foreign Application Priority Data
Aug. 30, 1993 (DE) P 43 29 177.5

(51) Int. Cl. C12N 9/12; C07H 21/04; C12P 21/04; C12N 5/06; C07K 16/40

(52) U.S. Cl. 435/194; 435/69.1; 435/320.1; 435/325; 435/70.21; 530/388.26; 536/23.2

(57) ABSTRACT

The invention relates to a PLK protein,

which is characterized in that it comprises

(a) the amino acid sequence shown in SEQ ID No: 1 or

(b) variants of the sequence from (a).

### DESCRIPTION

The present invention relates to a new serine threonine kinase, the gene coding therefor and the detection of changes in this gene and its products in human tumours.

The activation of intracellular biochemical networks as a response to external stimuli leads to coordinated control of growth and of differentiation in eukaryotes. Protein kinases are known as constituents of many signal transduction pathways. In this connection, these kinases phosphorylate their normal physiological substrates and are regulated in their enzymatic activity by interaction with other kinases and phosphatases. The identification of a large number of protein kinases in many eukaryotic cells of mammals, yeast and Drosophila makes it probable that fundamental cellular differentiation and growth processes are controlled by identical mechanisms in a wide spectrum of organisms.

In eukaryotes, all protein kinases known until now phosphorylate the hydroxyamino acids serine, threonine or tyrosine. Phosphorylation by these kinases plays a prominent role in the control of mitosis and cellular differentiation. Receptors for numerous polypeptide growth factors are transmembrane tyrosine kinases which, for their parts, phosphorylate serine/threonine kinases such as protein kinase C, MP kinase and p74raf. The central component of the cell cycle machinery is the serine/threonine kinase p34cdc2, which has originally been isolated as a product of the mitosis gene cdc2 (cell division cycle) from Schizosaccharomyces pombe and CDC28 from Saccharomyces cerevisiae. The activity of p34cdc2 is regulated by interaction with cyclins. In the G1 phase, the start of the cell cycle, p34cdc2 is not associated with cyclins and has no kinase activity. If cells are supplied with sufficient nutrients, G1 cyclin accumulates which, by association, initiates the kinase activity of p34cdc2. By this means “start” is exceeded and the change in the cell cycle (D)NA replication, formation of the MTOC, microtubule organizing centre) is initiated. In this connection, the synthesis of cyclin B also begins, which then binds to p34cdc2. This complex is inactive, since the binding of cyclin B induces the phosphorylation of p34cdc2 on tyrosine 15, whereby the kinase activity is inhibited. The inactive complex, which is also called preMPF (maturation promoting factor), is also phosphorylated on threonine 160. This phosphorylation is necessary for the MPF activity, but not sufficient in order to abolish the inhibitory effects of tyrosine phosphorylation. The subsequent dephosphorylation of p34cdc2 during the late G2 phase of the cell cycle activates MPF and leads to the induction of mitosis (M phase). The post-translational reactions, which underlie a complex physiological control, play an essential part in the temporal regulation of the MPF activity. The protein tyrosine kinase wee1 was originally isolated from Schizosaccharomyces pombe and inhibits, via the mechanism described, entry into mitosis, while the product of the cdc25 gene promotes the start of mitosis. Active MPF activates tyrosine phosphatase and inhibits protein tyrosine kinase, which modify p34cdc2, whereby MPF is completely activated explosively, such that cells are driven into mitosis very rapidly and irreversibly.

The most recent investigations of the cell cycle show that essential regulators of the cell cycle are involved in carcinogenesis. This is not surprising because constant proliferation of cells is an outstanding feature of tumours. Changes to cyclin A, which binds both to p34cdc2 and to p34cdc2-related protein kinase, can cause the transformation of cells. The cyclin A gene is the integration site for a fragment of the hepatitis B virus genome in a human hepatocarcinoma. Moreover, cyclin A is associated with the transforming protein E1A in adenovirus-transformed cells. Cyclin A is possibly a target protein of E1A, since it is associated in the S phase with the transcription factor E2F in a complex which has lower transcription activity than free E2F. A further constituent of this complex is p34cdc2. The connection with gene expression is produced in this way. E1A can destroy this complex, whereby E2F is released. Thus certain genes can be regulated which are important for the transformed phenotype.

Moreover, there are further relationships between oncoproteins, tumour suppressor gene products and cyclin-p34cdc2 complexes. The mos oncoprotein is likewise a serine/threonine kinase. The c-mos protein from Xenopus is a component of the cytostatic factor, which is necessary for the stabilization of the activated cyclin B-p34cdc2 in growth-inhibited Xenopus eggs. The role of the mos protein, however, is still unclear, as it does not appear to phosphorylate the complex in vivo. On the one hand, c-mos stabilizes the activated cyclin B-P34cdc2 complex, whereby the mitosis activity of the cells is inhibited, and on the other hand it promotes the cell cycle. Previous investigations confirm the assumption that the differing behaviour of the cell cycle machinery is controlled by the amount of mos protein.

Various oncoproteins such as the src and abl protein tyrosine kinases are likewise phosphorylated by the serine/threonine kinase p34cdc2 in the context of mitosis. In the src family, mitotic phosphorylation is accompanied by increased kinase activity. The products of the tumour suppressor genes RB and p53 likewise form complexes with cyclin-p34cdc2 and are phosphorylated in this process. In the case of RB, the phosphorylation appears to be necessary in order to inactivate the RB function so that the cells of G1 can progress into the S phase. As a consequence of the aberrant expression of the cyclin-p34cdc2 complex, it follows hat such tumour suppressor gene products are preserved in their phosphorylated inactive state, which results in unchecked cell division.

The connection between the function of serine/threonine kinases of the cell cycle and the transformation shows clearly that slight disturbances in the mitosis processes play a critical part in carcinogenesis. As carcinogenesis appears to be a multi-stage process, mutations in cell cycle regulators cooperate with mutations which activate protooncogenes or inactivate tumour suppressor genes.

On account of the enormous clinical importance of serine threonine kinases, there was thus a need to make available a new serine threonine kinase and the gene coding therefor.

The invention thus relates to a new serine threonine kinase called PLK, which is characterized in that it

(a) comprises the amino acid sequence shown in SEQ ID No: 2 or

(b) variants of the sequence from (a).

Preferably, the PLK protein according to the invention is a protein obtainable from man, i.e. it is the protein shown in SEQ ID No. 1 and No. 2 or a naturally occurring human variant thereof.

The invention also relates to a new protein which comprise parts of the amino acid sequence shown in SEQ ID No: 1 and 2. The invention preferably relates to a PLK protein which contains the amino acid sequence shown in SEQ ID No: 1 and 2; however, it can also contain variants of this sequence. The term “variants” within the meaning of the present invention is understood as meaning sequences which differ as a result of substitution, deletion and/or insertion of individual amino acids or short amino acid sections from the amino acid sequence shown in SEQ ID No: 1 and 2.

The term “variants” includes both naturally occurring allelic variations of the PLK protein, as well as proteins produced by recombinant DNA technology (in particular by in vitro mutagenesis with the aid of chemically synthesized oligonucleotides), which correspond with respect to their biological and/or immunological activity to the protein shown in SEQ ID No: 1.

Proteins according to the invention are preferably distinguished in that on the amino acid level they have a homology of at least 95%, compared with the amino acid sequence shown in SEQ ID No: 1 and 2.

The gene PLK (polo-like kinase) described here and coding for the protein according to the invention was isolated from a cDNA bank, based on human lung tumour RNA (squamous cell carcinoma).

Expression of the PLK Gene in Proliferating Cells and Tissues

For a Northern blot investigation, RNA was isolated from the following human adult tissues: lung, liver, heart, brain, pancreas, kidney, placenta, skeletal muscle, oesophagus, colon, stomach and spleen. Only in placenta and colon, i.e. in tissues which contain a greater percentage of proliferating cells, was PLK expression detected. The length of the PLK-mRNA is about 2.3 kb. In the following, the hypothesis was investigated that PLK expression correlates with the proliferation of cells: for this, human tumours including surrounding normal reference tissue were investigated. Strong expression was found in tumours of the following organs: lung, breasts, oesophagus, stomach and intestine as well as in leiomyosarcomas and non-Hodgkin lymphomas. In a random sample group of 48 lung tumours, PLK was strongly expressed in 84% of the samples. In the remaining 16% of the tumours, PLK was expressed slightly or not at all. Southern blot investigations of restricted DNA from tumours and healthy reference tissues showed no differences with a PLK-specific sample.

Table 1 gives a breakdown by means of the tumour type and the PLK expression status:

TABLE 1

| | positive | negative |
|---|---|---|
| Lung: | | |
| Squamous cell carcinoma: | 24 | 2 |
| Adenocarcinoma: | 10 | 2 |
| Large-cell carcinoma: | 6 | 1 |
| Small-cell carcinoma: | 3 | 0 |
| References (adult human lung): | 0 | 48 |
| Liver metastasis: | 1 | 0 |
| Leiomyosarcoma: | 1 | 0 |
| Non-Hodgkin lymphoma: | 1 | 0 |
| Breast: | | |
| Breast carcinoma: | 7 | 1 |
| References (normal breast tissue): | 0 | 8 |
| Intestine: | | |
| Colorectal carcinoma: | 3 | 0 |
| References (normal colon tissue): | 3 | 0 |

In addition to human tissues and primary cells, the following cell lines were tested for

PLK expression:

HELA cell line (cell type: cervix carcinoma),

lung epithelium cell line (cell type: lung carcinoma),

A431 cell line (cell type: epidermoid carcinoma),

HEP-38 cell line (cell type: hepatocellular carcinoma),

A498 cell line (cell type: kidney carcinoma),

BT 20 cell line (cell type: breast carcinoma),

T47D cell line (cell type: ductal breast carcinoma),

SKBR3 cell line (cell type: adenocarcinoma of the breast),

NCF7 cell line (cell type: adenocarcinoma of the breast) and

HUV-EC-C cell line (cell type: umbilical cord vein endothelial cells).

PLK transcripts were detectable in all cell lines investigated.

It was found that the expression of the PLK gene correlates with the proliferation activity. The possibility thus results of employing the amount of PLK gene products (mRNA or protein) as a measure of the division activity of a cell and thus as a proliferation marker for the analysis of human neoplasias. As emerges from Table 1, an increased PLK expression is detectable in a very high percentage of the tumours investigated. PLK is thus a universally employable proliferation marker.

Mitogen-Induced Expression of PLK in Lymphocytes

Resting lymphocytes from peripheral blood do not express the PLK gene. Addition of PHA (phytohaemaglutinin) or PHA/IL-2 (interleukin-2) stimulates lymphocytes and the expression of PLK is induced. The expression of PLK can therefore also be taken as a measure of the activation of lymphocytes. In a further experiments the cell line A431 was first cultured in serum-containing and then in serum-free medium. The expression of PLK decreased in the course of the 5 days without serum. After addition of serum, PLK expression increased strongly again.

Decrease of PLK Expression in Human Macrophages

Macrophages from peripheral blood are cells having, if at all, very low proliferation activity. Depending on the donor, macrophages after 15 days in culture had no or only very low PLK expression. After addition of LPS (lipopolysaccharide), the PLK expression was switched off completely within 24 hours.

Correlation of PLK Expression with the Prognosis of Tumour Patients

It was furthermore found that the prognosis, in particular the life expectancy of tumour patients, correlates with the expression of PLK For this, a relatively large collective of lung tumour patients was investigated and it was found that with increasing PLK expression the life expectancy of the patients falls. PLK can thus also be employed as a diagnostic tumour marker for the prognosis. The special feature of PLK can be seen in the fact that an increased expression occurs in more than 80% of the patients investigated, so that PLK is a very good tumour marker which indicates the presence of tumours with high probability.

The capability of PLK to be employed as a prognosis marker applies not only to lung tumours, but also to other tumours such as breast cancer, etc.

The invention thus relates to a process for the determination of the mitotic activity of cells, in particular of human cells, in which the expression potency of the gene coding for the PLK protein is determined in the cell concerned, e.g. on the transcription plane by Northern blot processes and/or on the protein plane by activity determinations or immunological methods. On account of the correlation of the PLK expression shown above in human tissues, primary cells and cell lines with the mitotic activity of the cells, the expression potency of the PLK gene represents a suitable variable in order to determine the status of specific cells with respect to mitotic activity. This process, which can be carried out extracorporeally, can be of great use, for example, in the field of tumour diagnosis or immunodiagnosis.

The amino acid sequence shown in SEQ ID No: 1 and 2 represents the total PLK protein. This protein has 603 amino acids. The PLK gene or variants thereof can be cloned routinely in a vector, such that this vector is brought into a suitable host cell for expression, the protein according to the invention being formed. Preferred host cells are microorganisms such as E. coli or yeast, but also higher cells (e.g. mammalian or insect cells). Preferred expression vectors are, for example, plasmids, bacteriophage lambda for prokaryotes, yeast vectors or viral vectors for higher cells (e.g. SV40, Vaccinia, Baculoviruses). With respect to the expression of the PLK gene, reference should be made in particular to the methods mentioned in Sambrook et al., (Molecular Cloning, A Laboratory Manual (1989) Cold Spring Harbor Laboratory Press). Furthermore, the expression of related kinase proteins is described in the works of Crews, C. M. et al., (1991), Proc. Natl. Acad. Sci. USA 88, 8845-8849; Ben-David, Y. et al., (1991) EMBO J. 10 (2), 317-325; Parker, L. L. et al., (1991) EMBO J. 10 (5), 1255-1263; Parker L. L. et al., (1992) Science 257, 1955-1957; Colemann, T. R. et al., (1993) Cell 72, 919-929, to whose disclosure reference is made here for this purpose.

A comparison of the PLK sequence with the EMBL databank yields a high measure of homology with the family of serine/threonine kinases. The protein according to the invention is called PLK (polo-like kinase) on account of its homology to the polo gene, which was isolated from Drosophila melanogaster.

The following protein kinase-specific sequence characteristics occur.

1. The ATP binding pattern:

GlyLysGlyGlyPheAla . . . . . (16 amino acids) . . . . Lys (amino acids 60-86)

2. Two amino acid patterns lying in the catalytic domain of protein kinases, which are highly conserved in all protein kinases with respect to the amino acid sequence and the distance of the ferns from one another:

| | |
|---|---|
| HisArgAspLeu | (AA 174-177) |
| AspPheGly | (AA 194-196) |

The present invention furthermore relates to a nucleic acid which codes for a PLK protein according to the invention or parts thereof. This nucleic acid can be, for example, genomic DNA or RNA. Preferably, however, it is in this case a recombinant DNA molecule.

The invention furthermore relates to a nucleic acid according to the invention, which

(a) contains the coding sequence shown in SEQ ID No: 1,

(b) a nucleic acid sequence corresponding to the sequence from (a) in the context of the degeneration of the genetic code or

(c) a sequence hybridizer with the sequences from (a) and/or (b) under stringent hybridization conditions.

Under stringent hybridization conditions within the meaning of the present invention is understood as meaning that a hybridization still occurs even after washing at 55° C., preferably at 62° C., particularly preferably at 68° C., in an aqueous low salt buffer (e.g. 0.2×SSC, 0.1% SDS) (see also Sambrook J. et al., (1989), Molecular Cloning. A Laboratory Manual).

The invention also relates to nucleic acids which contain a section at least 20 nucleotides long of the sequence shown in SEQ ID No.: 1. Preferably, this section has a nucleotide sequence specific for the PLK gene. These nucleic acids are suitable, in particular, for the production of antisense nucleic acids which can be employed therapeutically.

Additionally, the invention further relates to a vector which contains at least one copy of a nucleic acid according to the invention or a part thereof. The vector can be replicable in eukaryotes or prokaryotes. It can be a vector which can be integrated into the genome of the host cell (e.g. bacteriophage lambda) or a vector which is present extrachromosomally (e.g. a plasmid). The vector according to the invention can be obtained by subcloning of the PLK gene in a base vector. Base vectors of this type, in particular vectors containing the elements necessary for protein expression, are familiar to a person skilled in the art.

The present invention further relates to a cell which is transformed using a nucleic acid according to the invention or a vector according to the invention. The cell can be either a eukaryotic or a prokaryotic cell. Processes for the transformation of cells are general prior art and therefore do not need to be explained.

The present invention likewise relates to the use of the PLK protein or of the gene coding therefor or of an antibody directed against the protein in tumour diagnosis. Experiments have shown that the expression of the PLK gene in tumours and corresponding tumour-free reference tissues is different. There is therefore the possibility to draw conclusions on the mitotic activity of cells from the level of expression of the gene or of the protein.

The invention additionally relates to a diagnostic or therapeutic composition based on a PLK protein, on an antibody directed against it or a nucleic acid according to the invention (e.g. as an antisense nucleic acid for the suppression of gene expression). A composition of this type can optionally contain known pharmaceutical diluents, fillers, excipients and auxiliaries.

It was possible to show the inhibition of the growth of cells by inhibition of PLK expression experimentally by antisense constructs and by dominant-negative mutants. A particularly preferred aspect of the present invention is therefore the gene therapy application of the nucleic acids according to the invention, in which these nucleic acids are administrated to a patient in replicable form (e.g. on a vector suitable for integration into the genome), e.g. in order to produce antisense nucleic acids within the cell. Alternatively, the antisense nucleic acids can also be introduced directly into the cell (e.g. by microinjection).

The invention further relates to a method for the determination of the activity of lymphocytes, in which a protein according to the invention, a nucleic acid according to the invention or an antibody directed against the protein is used. This method can be carried out, for example, extracorporeally on taken blood samples. This process is particularly suitable for the detection of disorders of the immune system, in particular in autoimmune disorders or immunodeficiency syndromes including AIDS.

Finally, the present invention additionally comprises the use of the PLK protein or of fragments of this protein as an immunogen for the production of antibodies. The production of antibodies against the PLK protein is carried out in a customary manner by immunization of experimental animals with the complete PLK protein or fragments thereof and subsequent recovery of the resulting (polyclonal) antibodies. According to the method of Köhler and Milstein or its further developments, monoclonal antibodies can be obtained from the PLK antibody-producing cells of the experimental animals in a known manner by cell fusion. Human monoclonal antibodies can also be obtained.

SEQ ID No. 7 shows a nucleic acid sequence 2503 bp long, which contains the region attaching directly to the 5′-side on the start codon of the PLK gene. It was possible to detect by chloramphenicol transferase (CAT) assays, that the indicated sequence includes the promoter of the PLK gene. The starting point of the transcription in SEQ ID No: 7 is at nucleotide 2460. Positive-regulatory element of the promoter are found within the region between the nucleotides 184 and 676, and 1163 and 2503. Negative-regulatory elements are found within the region between the nucleotides 676 and 1163. It was possible to identify these regulatory elements with the aid of CAT assays using deletion clones. By sequence comparison, it was furthermore found that a CCAAT box is localized between the nucleotides 2407 and 2411 and two potential SP1 binding sites are localized between the nucleotides 2375-2384.

The PLK promoter has the property that it is only active in proliferating cells. It can therefore also be employed in combination with a gene which is toxic for the cells (e.g. the gene for diphtheria toxin or the gene for cholera toxin) for the gene therapy treatment of tumours. The PLK promoter in combination with the PLK gene in the antisense orientation offers a further possibility of therapeutic use. In this manner, a selective inhibition of the growth of dividing cells can be achieved.

The present invention thus also relates to a nucleic acid which contains the sequence shown in SEQ ID No: 7 or a section thereof, preferably a section having identical biological activity. This nucleic acid can be, for example, in operative linkage with a toxic gene or with the PLK gene in the antisense orientation or a section thereof which is preferably longer than 20 nucleotides. These nucleic acids can be used, in particular, for gene therapy.

The invention is intended to be explained further by the sequence protocol and figures SEQ ID No: 1 to SEQ ID No: 7 and FIG. 1 together with the following examples, without it being intended here to restrict the scope of the invention.

SEQ ID No: 1 shows a 2124 bp-long nucleic acid sequence, which contains the genetic information coding for the PLK gene,

SEQ ID No: 2 shows the 603 AA-long amino acid sequence of the PLK protein,

SEQ ID No: 3 shows the nucleic acid sequence of the oligonucleotide primer P6 DEA

SEQ ID No: 4 shows the nucleic acid sequence of the oligonucleotide primer Eco HRDL

SEQ ID No: 5 shows the nucleic acid sequence of the oligonucleotide prime P12T+,

SEQ ID No: 6 shows the nucleic acid sequence of the oligonucleotide primer RAF2 and

SEQ ID No: 7 shows the 2503 bp-long nucleic acid sequence of the PLK promoter and

FIG. 1 shows the post-operative survival rate of lung cancer patients as a function of PLK expression.

### EXAMPLE 1

Isolation of a Part Sequence of the PLK Gene

1.1 RNA was isolated from human lung tumour tissue according to the method of Chirgwin et al., (Biochemistry 18 (1979), 5294). Using this RNA, an oligo(dT)-mediated cDNA synthesis was carried out. The reaction conditions were as follows:

50 mmol/l of tris/HCl, pH 8.3 (22° C.)

75 mmol/l of KCl

10 mmol/l of dithiothreitol DTT)

3 mmol/l of MgCl₂

500 μmol/l of dNTP solution

0.2 μmol/l of oligonucleotide primer P12T+having the nucleic acid sequence shown in SEQ ID No: 5

3.0 μg of total RNA

100 μg/ml of bovine serum albumin

10 units of reverse transcriptase (from Moloney murine leukaemia virus)

The above reaction mixture was incubated at 37° C. for one hour in a reaction volume of 20 μl.

1.2 The cDNA from 1.1 was amplified using two primers Eco HRDL and P6 DEA complementary to two too-highly conserved regions of PTK genes. The nucleic acid sequence of the primer P6 DEA is indicated in SEQ ID No: 3. The sequence of Eco HRDL is shown in SEQ ID No: 4 (this sequence is homologous to the nucleotides 588 to 601 of the sequence shown in SEQ ID No: 1).

Reaction conditions:

8.3 mmol/l of tris/HCl, pH 8.8 (220° C.)

41.7 mmol/l of KCl

1.25 mmol/l of MgCl₂

0.01% of gelatin

166.7 μmol/l of dNTP solution

0.6 μmol/l of primer P6 DEA and Eco HRDL

5 units of Taq polymerase

10 μl of cDNA synthesis mixture

The reaction volume was 60 μl. The conditions for a PCR cycle were as follows:

95° C. denaturation for 1 minute,

40° C. hybridization for 2 minutes,

72° C. elongation for 3 minutes.

A total of 40 cycles were carried out.

The amplificate formed was set separated electrophoretically in a 2% strength agarose gel for the purpose of purification and the DNA in the length region from about 190 bp to 220 bp was electrically eluted and amplified again for 30 cycles under the conditions indicated above. The PCR products thus obtained and purified were then digested using the restriction endonuclease EcoRI and cloned into the Bluescript-KS vector (Stratagene) which was likewise cleaved with EcoRI.

In this manner, a 199 bp-long DNA fragment was isolated and sequenced (Sambrook J. et al., Molecular Cloning, A Laboratory Manual, Cold Spring Harbor Laboratory 1989). The nucleic acid sequence of this DNA fragment corresponds to the nucleotides 588 to 787 of the sequence shown in SEQ ID No: 1.

### EXAMPLES 2

Isolation of a PLK-cDNA

A cDNA bank from poly A+ RNA of human lung tumour tissue was established using 1.8×10⁶ recombinant clones. The isolation of the mRNA was carried out according to Aviv and Leder (Proc. Natl. Acad. Sci. USA 69 (1972), 1408). The cDNA cloning was carried out according to Gubler and Hoffman (Gene 25 (1983), 263). This gene bank was inspected by means of a DNA probe. The DNA probe was prepared by PCR The oligonucleotide RAF2 was used as a primer for the PCR. The nucleic acid sequence for RAF2 is shown in SEQ ID No: 6 (this sequence corresponds to the nucleotides 746 to 767 of the sequence shown in SEQ ID No: 1).

Reaction conditions:

20 ng of PLK insert (from Example 1)

10 mmol/l of tris/HCl, pH 8.8 (22° C.)

50 mmol/l of KCl

1.5 mmol/l of MgCl₂

0.01% of gelatin

0.8 μmol/l of α³²P-dCTP (6000 Ci/mmol)

0.8 μmol/l of dATP/dGTP/dTTP

0.2 mol/l of the primer RAF2

2.5 units of Taq polymerase

The reaction volume was 25 μl. The reaction was carried out in 20 cycles under the PCR conditions mentioned above (in Example 1).

For the detection of a positive clone, cDNA from the gene bank was immobilized on filters and hybridized on the radiolabelled DNA probe (1×10⁶ dpm/ml of solution at a specific probe activity of 5×10⁹ dpm/μg of DNA). The hybridization temperature was 42° C.

Hybridization solution:

5×SSC

0.02 mol/l of tris/HCl, pH 7.6 (22° C.)

1× Denhardt solution

10% of dextran sulphate

0.1% of SDS

100 μg/ml of herring sperm DNA

50%/o of formamide

The washing solution employed was 2×SSC, 0.1% SDS at a washing temperature of 42° C.

A positive clone was found and characterized. It contained the nucleic acid sequence shown in SEQ ID No: 1.

### EXAMPLE 3

Inhibition of PLK Expression

1×10⁴ A431 cells were seeded and allowed to grow for 2 to 3 days to a confluence of 95%. The culture was then kept for 24 hours in RPMI 1640 medium containing 0.5% foetal calf serum. 1 to 2×10⁻¹² ml of a liquid containing 2 μg/μl of TKF antisense RNA per cell were injected by microinjection. This TKF antisense RNA was prepared using T3 or T7 RNA polymerase by transcription of the sequence shown in SEQ ID No: 1 in reverse orientation.

The cells were kept further in RPMI 1640 medium containing 0.5% or 10% foetal calf serum. After 18 hours, the cells were pulsed with ³H-thymidine (0.5 μCi/ml; Amersham) for 3 to 4 hours. After washing with phosphate-buffered saline solution (PBS), fixing was carried out in 3.5% formaldehyde-PBS, then layering with film emulsion (NTP-2, Kodak) and incubation for 48 hours for the purpose of development. The cells were stained according to Giemsa, counted and photographed. It was shown that it was possible to inhibit the PLK expression and the growth of cells by addition of the antisense RNA.

### EXAMPLE 4

PLK as a Diagnostic Tumour Marker

It was found that in operated lung tumour patients there is a clear correlation of PLK expression with the survival time of the patient. It was found that patients with high PLK expression die significantly earlier than those in the comparison group having low PLK expression

The results of this experiment are indicated in the following Table 2. For the analysis, of PLK expression, the mRNA from the tumour tissue of the respective patient was investigated by means of a Northern blot analysis. The values indicated in Table 2 are standardized with respect to the expression of actin mRNA. The survival time of the patients after the operation is indicated in months. The symbol “^{•}” means that the patient was still alive at the time indicated

TABLE 2

| | | | | Post- | |
|---|---|---|---|---|---|
| | | | | operative | |
| Patient | Age/ | Tumour | | survival | PLK |
| No. | sex | stage | Tumour | time | expression |
| | | | Squamous cell | | |
| | | | carcinoma | | |
| 1 | 71 m | II | | 10 | 3.79 |
| 2 | 71 m | III | | *28 | 0.48 |
| 3 | 60 m | III | | 5 | 1.02 |
| 4 | 54 m | III | | 19 | 1.12 |
| 5 | 61 m | I | | 17 | 0.36 |
| 6 | 67 m | ? | | 24 | 0.15 |
| 7 | 70 m | I | | 35 | 0.90 |
| 8 | 69 m | II | | *41 | 0.08 |
| 9 | 72 m | I | | *37 | 0.67 |
| 10 | 67 m | III | | *55 | 0.35 |
| 11 | 66 m | II | | 10 | 0.13 |
| 12 | 57 m | I | | *31 | 0.10 |
| 13 | 61 m | I | | 4 | 6.94 |
| 14 | 64 m | I | | 7 | 1.96 |
| 15 | 42 m | II | | 27 | 0.28 |
| 16 | 70 m | I | | 0 | 3.84 |
| 17 | 54 m | II | | 25 | 4.66 |
| 18 | 61 m | II | | 12 | 1.99 |
| 19 | 67 m | I | | 7 | 1.14 |
| 20 | 70 m | II | | 0 | 3.75 |
| 21 | 53 f | I | | *38 | 1.41 |
| 22 | 36 m | II | | *37 | 8.57 |
| 23 | 75 m | I | | 4 | 2.01 |
| | | | Adenocarcinoma | | |
| 24 | 77 f | I | | *38 | 0.31 |
| 25 | 56 m | II | | *32 | 0.73 |
| 26 | 56 m | III | | 13 | 2.14 |
| 27 | 71 m | III | | 2 | 13.1 |
| 28 | 72 m | I | | *32 | 1.04 |
| 29 | 54 m | III | | 14 | 0.94 |
| 30 | 60 m | I | | 26 | 1.34 |
| 31 | 70 m | I | | 38 | 0.96 |
| 32 | 43 m | I | | *32 | 4.12 |
| | | | Large-cell | | |
| | | | carcinoma | | |
| 33 | 62 m | III | | 26 | 3.97 |
| 34 | 66 m | I | | *44 | 14.8 |
| 35 | 66 f | III | | 2 | 32.3 |
| 36 | 64 m | I | | 2 | 11.0 |
| 37 | 40 m | I | | *36 | 17.9 |
| 38 | 53 f | III | | 0 | 5.50 |
| | | | Mixed-cell | | |
| | | | carcinoma | | |
| 39 | 61 m | I | | 4 | 0.10 |
| 40 | 57 m | II | | 0 | 3.34 |
| 41 | 49 m | I | | 3 | 0.33 |
| | | | Broncheoalveolar | | |
| | | | carcinoma | | |
| 42 | 76 f | I | | *43 | 0.40 |
| 43 | 77 f | I | | *44 | 0.12 |
| 44 | 77 f | II | | *48 | 0.02 |
| 45 | 57 m | | | *42 | 0.12 |
| | | | Small-cell | | |
| | | | lung cancer | | |
| 46 | 77 m | III | | 13 | 0.55 |
| 47 | 62 m | I | | 10 | 6.23 |
| 48 | 63 m | III | | 18 | 11.2 |
| 49 | 58 m | I | | *35 | 11.2 |
| 50 | 63 m | II | | 13 | 0.04 |
| | | | Leiomyosarcoma in | | |
| | | | the lung | | |
| 51 | 46 m | III | | 12 | 0.42 |

FIG. 1 shows the associated statistical Kaplan-Meier assessment of the patient data (E. L. Kaplan and P. Meier, J. Am. Stat. Assoc. 53 (1958), 457-481). Curve 1 shows the operative survival rate of 23 lung cancer patients with an absolute PLK expression >1 standardized on actin). Curve 2 shows the survival rate of 28 lung cancer patients having an absolute standardized PLK expression <1. It is evident from FIG. 1 that patients having high PLK expression die significantly earlier than those in the comparison group having low PLK expression.

1. PLK protein, characterized in that it comprises
(a) the amino acid sequence shown in SEQ ID No: 2 or
(b) variants of the sequence from (a).

2. Protein according to claim 1, characterized in that it is obtainable from man.

3. Nucleic acid, characterized in that it codes for a PLK protein according to claim 1 or 2.

4. Nucleic acid according to claim 3, characterized in that it is a recombinant DNA molecule.

5. Nucleic acid according to claims 3 or 4, characterized in that it contains
(a) the protein-coding sequence shown in SEQ ID No: 1,
(b) a nucleic acid sequence corresponding to the sequence from (a) in the context of the degeneration of the genetic code or
(c) a sequence hybridizing with the sequences from (a) and/or (b) under stringent hybridization conditions.

6. Nucleic acid, characterized in that it contains a section at least 20 nucleotides long of the sequence shown in SEQ ID No: 1.

7. Vector, characterized in that it contains at least one copy of a nucleic acid according to one of claims 3 to 6 or a part thereof.

8. Cell, characterized in that it is transformed using a nucleic acid according to one of claims 3 to 6 or a vector according to claim 7.

9. Use of a protein according to claim 1 or 2 or of fragments of this protein as an immunogen for the production of antibodies.

10. Use of a protein according to claim 1 or 2, of a nucleic acid according to one of claims 3 to 6 or of an antibody against such a protein for tumour diagnosis and/or tumour therapy.

11. Use of a protein according to claim 1 or 2, of a nucleic acid according to one of claims 3 to 6 or of an antibody against such a protein for the production of a pharmaceutical composition for tumour diagnosis and/or tumour therapy.

12. Use of a protein according to claim 1 or 2 or of a nucleic acid according to one of claims 3 to 6 or of an antibody directed against such a protein for the determination of the activity of lymphocytes.

13. Use according to claim 12 for the determination of lymphocyte activity in disorders of the immune system, in particular in autoimmune disorder or immunodeficiency syndromes.

14. Use of a nucleic acid according to one of claims 3-6 for the production of a composition for gene therapy.

15. Diagnostic composition, characterized in that, as an active constituent, it comprises a protein according to claim 1 or 2 or an antibody against such a protein or a nucleic acid according to one of claims 3 to 6.

16. Medicament, characterized in that as an active constituent it comprises a protein according to claim 1 or 2 or an antibody directed against such a protein or a nucleic acid according to one of claims 3 to 6, optionally together with pharmaceutically customary auxiliaries, excipients, fillers and diluents.

17. Method for the determination of the mitotic activity of cells, in particular of human cells, characterized in that the expression strength of the gene coding for a protein according to claim 1 or 2 is determined in the cell concerned.

18. Process according to claim 17, characterized in that the expression strength is determined on the transcript and/or protein plane.

19. Nucleic acid, characterized in that it contains the sequence shown in SEQ ID No: 7 or a functional section thereof.

20. Nucleic acid according to claim 19, characterized in that it is in operative linkage with a toxic gene.

21. Nucleic acid according to claim 19, characterized in that it is in operative linkage with the PKL gene in the antisense orientation or a section thereof.

22. Use of a nucleic acid according to one of claims 19-21 for the production of a composition for gene therapy.


SEQUENCE LISTING (7 sequences)

SEQ ID NO: 1 - DNA (cDNA), 2124 bp, Homo sapiens; both-stranded, linear
  CDS 65..1873
  anti-sense: yes
ggggaggagcggagcggtgcggaggctctgctcggatcgaggtctgcagcgcagcttcgggagcatgagtgctgcagtgactgcagggaagctggcacgggcaccggccgaccctgggaaagccggggtccccggagttgcagctcccggagctccggcggcggctccaccggcgaaagagatcccggaggtcctagtggacccacgcagccggcggcgctatgtgcggggccgctttttgggcaagggcggctttgccaagtgcttcgagatctcggacgcggacaccaaggaggtgttcgcgggcaagattgtgcctaagtctctgctgctcaagccgcaccagagggagaagatgtccatggaaatatccattcaccgcagcctcgcccaccagcacgtcgtaggattccacggctttttcgaggacaacgacttcgtgttcgtggtgttggagctctgccgccggaggtctctcctggagccgcacaagaggaggaaagccctgactgagcctgaggcccgatactacctacggcaaattgtgcttggctgccagtacctgcaccgaaaccgagttattcatcgagacctcaagctgggcaaccttttcctgaatgaagatctggaggtgaaaataggggattttggactggcaaccaaagtcgaatatgacggggagaggaagaagaccctgtgtgggactcctaattacatagctcccgaggtgctgagcaagaaagagcacagtttcgaggtggatgtgtggtccattgggtgtatcatgtataccttgttagtgggcaaaccaccttttgagacttcttgcctaaaagagacctacctccggatcaagaagaatgaatacagtattcccaagcacatcaaccccgtggccgcctccctcatccagaagatgcttcagacagatcccactgcccgcccaaccattaacgagctgcttaatgacgagttctttacttctggctatatccctgcccgtctccccatcacctgcctgaccattccaccaaggttttcgattgctcccagcagcctggaccccagcaaccggaagcccctcacagtcctcaataaaggcttggagaaccccctgcctgagcgtccccgggaaaaagaagaaccagtggttcgagagacaggtgaggtggtcgactgccacctcagtgacatgctgcagcagctgcacagtgtcaatgcctccaagccctcggagcgtgggctggtcaggcaagaggaggctgaggatcctgcctgcatccccatcttctgggtcagcaagtgggtggactattcggacaagtacggccttgggtatcagctctgtgataacagcgtgggggtgctcttcaatgactcaacacgcctcatcctctacaatgatggtgacagcctgcagtacatagagcgtgacggcactgagtcctacctcaccgtgagttcccatcccaactccttgatgaagaagatcaccctccttaaatatttccgcaattacatgagcgagcacttgctgaaggcaggtgccaacatcacgccgcgcgaaggtgatgagctcgcccggctgccctacctacggacctggttccgcacccgcagcgccatcatcctgcacctcagcaacggcagcgtgcagatcaacttcttccaggatcacaccaagctcatcttgtgcccactgatggcagccgtgacctacatcgacgagaagcgggacttccgcacataccgcctgagtctcctggaggagtacggctgctgcaaggagctggccagccggctccgctacgcccgcactatggtggacaagctgctgagctcacgctcggccagcaaccgtctcaaggcctcctaatagctgccctcccctccggactggtgccctcctcactcccacctgcatctggggcccatactggttggctcccgcggtgccatgtctgcagtgtgccccccagccccggtggctgggcagagctgcatcatccttgcaggtgggggttgctgtataagttatttttgtacatgttcgggtgtgggttctacagacttgtccccctccccctcaaccccaccatatgaattgtacagaatatttctatt

SEQ ID NO: 2 - protein, 603 aa
MSAAVTAGKLARAPADPGKAGVPGVAAPGAPAAAPPAKEIPEVLVDPRSRRRYVRGRFLGKGGFAKCFEISDADTKEVFAGKIVPKSLLLKPHQREKMSMEISIHRSLAHQHVVGFHGFFEDNDFVFVVLELCRRRSLLEPHKRRKALTEPEARYYLRQIVLGCQYLHRNRVIHRDLKLGNLFLNEDLEVKIGDFGLATKVEYDGERKKTLCGTPNYIAPEVLSKKEHSFEVDVWSIGCIMYTLLVGKPPFETSCLKETYLRIKKNEYSIPKHINPVAASLIQKMLQTDPTARPTINELLNDEFFTSGYIPARLPITCLTIPPRFSIAPSSLDPSNRKPLTVLNKGLENPLPERPREKEEPVVRETGEVVDCHLSDMLQQLHSVNASKPSERGLVRQEEAEDPACIPIFWVSKWVDYSDKYGLGYQLCDNSVGVLFNDSTRLILYNDGDSLQYIERDGTESYLTVSSHPNSLMKKITLLKYFRNYMSEHLLKAGANITPREGDELARLPYLRTWFRTRSAIILHLSNGSVQINFFQDHTKLILCPLMAAVTYIDEKRDFRTYRLSLLEEYGCCKELASRLRYARTMVDKLLSSRSASNRLKAS

SEQ ID NO: 3 - DNA (genomic), 29 bp; single-stranded, linear
  hypothetical: yes
  anti-sense: no
ttggaattcatcccnnnnnnccacacatc

SEQ ID NO: 4 - DNA (genomic), 24 bp; single-stranded, linear
  hypothetical: yes
  anti-sense: no
tttggaattcgtncaymgngayyt

SEQ ID NO: 5 - DNA (genomic), 37 bp; single-stranded, linear
  hypothetical: yes
  anti-sense: no
gcagaattcgtgaactgcggccgcatttttttttttt

SEQ ID NO: 6 - DNA (genomic), 22 bp; single-stranded, linear
  hypothetical: yes
  anti-sense: no
cacctcgaaactgtgctctttc

SEQ ID NO: 7 - DNA (cDNA), 2503 bp, Homo sapiens; both-stranded, linear
  hypothetical: no
  anti-sense: no
ctaacgggtgtggtggtgtgcacctgtagtcccagctactcgggaggctgaggcaggagaatcgcttgaacccaggaggtggaggctgcagtgagccaagatcacgccactgcactccagcctgggtgacagagcaagactccatctcaacaacaacaacaaaaaggaattaaggaggaagccacctggcttcctaagatagactttatggttaatgggatttattttagctggttaagtccaggacccctaccaggaagaactgaggtccatatttgaggttggtctcacccttttgcatgtggtcctactaataataattttgctgtaaagaagcctcaataaggagatggactgtgtgaaaagagatgagaaagcaggcctgtttgcaaggctggcccttggctggcatctgaaagcttggatttcaggagggttctcattactccctaaatgataagagtggctcactgtgcctaaactgttcgtacaaacaatgtgggttatgcccaacatctgctttccttctgggagtcagattttcatatgtgttaggcagaggatgaggtgagcagctcccaatgagaaccctgaacactgagtctgtaatgagcttcccttgtatacaacattgcacatgggttgtcacaactgattgctggaggaattgtgtcctatgtgactctgctgggagaggactgtgggaggcttacacctggtttccctggactttgtccatgcgcttttttcctttgctgattttgcttcctagcctttcgctgtagtaaaacatagccatgagtatgactacaggctgagtctgtgaatctcctagtacatcatcagactaggaggtggtggtgttggcaccccccagcacagggcacaagggagacttgcagggtgtttgtcatgttccctttctcaatctgggtgtggtttacagatttgttcagtttgtgaaaattcactgagctctatgaacaattataatatgtacatttttttctgtaagtgtattaaatttcaataaaaagaattcacactataggggtacaaaataacttaatttttcaaagtcttccttttattcagccaacattttagtaagtacccttttttttttaccaagcactgctctgggagcttgggacatggggtagaaaggacggcatcattgtcctcattgtccttcagcttagctgcaaggcaggtgaaggatgcttatagtatggttgatttttgctgggctctatgaaggatcagagggtgctaaggaggcacagagaaagagcttttaacgtgggtattttactgttccagggttgccactcaagtacctacagaacaaacgggtgtagctgtaaactgtggcaaactgaaagcatgtaggcacagagtaagtagctagagctgatttttttcaggaaaggccacaaatcccaacttttatgagctcctcttttaaatgctgtaaatgttttacaatggcggggcgcggtggctcacgcctgtaatcccagcatttgggaggccgaggggcagatcacttgaggtcaggagttcaagaccagcctggccaacatggtgaaacctcttctctactaaaattacaaaaattagccgggcatggtggtgcatgcttgtaattccagctgctagggaggccgaggcgggaggattgcttgaacccgggaagcagaggttgcagtgagctgagatcgtgccactgcactccagcctgggcgacagagcaagattccgtcacacacacaaaaaaaaggcgtggggggaggccaaacaaaaccccgcaagacacatttggctatgacctgccagtttgctaggcattcttccaaccttccctccctctgaccaagaaactgagtgtccactattttaggccctgggaaattcagtagcgaggaggccagacagcttcgttgcatcatggggggctctggtactgtgcttctccaacttcaggatgtgtaggaatcacctgagcagtcttgttgagaggcggacactgactcgggaggtctggggtagggcctgaacgtttgcctttgcggttctaacaagctctcaggtgatggcgatgctactgttccctggccccgaggtagaggaagatttaagtaaaagcttcctggaggaggcgcaagtgaaccgcaggagctttcccggacgcccgagaagggagaaaccccgaaggaattcctcctctctcggggctgggtctccgcatccacgccgggtttggtttcccaggctatcccacgtgttcgggcgtccgtgtcaatcaggttttccccggctgggtccgggtttaaaggctgctgctgcgcagggcgctcccatggtgccgcgcggcgggcgggtttggattttaaatccccgcggccaatcagtggcgcgcaggcttttgtaacgttcccagcgccgcgtttgaattcggggaggagcggagcggtgcggaggctctgctcggatcgaggtc